(19) United States
(12) Patent Application Publication
(10) Pub. No.: US 2002/0160051 A1
(43) Pub. Date: Oct. 31, 2002

(54) Homeopathic medicine comprising one or several mother-of-pearl constituents including bio-aragonite

(76) Inventor: Jean-Pierre Lablanchy, Paris (FR)

Correspondence Address: James C. Lydon, Suite 100, 100 Daingerfield Road, Alexandria, VA, 22314, US

(21) Appl. No.: 09/791,663

(22) Filed: Feb. 26, 2001

Related U.S. Application Data
(63) Continuation of application No. PCT/FR99/02048, filed on Aug. 26, 1999.

(30) Foreign Application Priority Data
Aug. 27, 1998 (FR) 98/10857

(51) Int. Cl. A61K 35/56

(52) U.S. Cl. 424/547

(57) ABSTRACT

A homeopathic medicine which includes at least biocompatible mother-of-pearl bio-aragonite. The medicine is used for treating pain of bone origin and in particular related to bone diseases cause by a pathology, pre-menopause or post-menopause estrogenic deficiency, ageing, cancer or trauma and to cartilaginous disorders, particularly osteoarthritis.

### TECHNICAL FIELD

The present invention relates to a homeopathic medication comprising one or several mother-of-pearl constituents, including bio-aragonite. It also relates to a process for manufacturing this medication.

### BACKGROUND ART

Within the context of the present invention, the expression “homeopathic medication” refers to a medication that is administered at low doses, in comparison with those administered using allopathy techniques, that is, with minimal dilution of the mother tincture of 1DH (one tenth potency).

Mother-of-pearl, or chonchylifere aragonite, is a biogenous mineralized formation, comprised mainly of a mineral part consisting of crystallized calcium carbonate in the form of aragonite and essentially of an organic matrix of fibrous and non-fibrous substances, representing approximately 1 to 2% of the total weight. The mother-of-pearl is obtained from marine mollusk shells, namely certain oysters such as Pinctada maxima, for which it makes up the most internal layer.

The document FR 2 742 661 discloses the use of compositions containing mother-of-pearl, with the view of making a pharmaceutical or cosmetological preparation intended to promote the synthesis of new cytokeratins.

Moreover, the properties of oyster shells in calcium supplementation and the very good intestinal absorption of oyster shell electrolysates are known, compared to that of calcium carbonate. (Takuo Fujita et all, Intestinal absorption of oyster shell electrolysate, Bone and Mineral, 4, p. 321-327 (1988)).

More precisely, it has been shown that in men, the oral absorption of oyster shells heated in a vacuum (“in vacuo”) enabled the bone mineral density of the backbone to be increased in a more significant manner than with a calcium carbonate-based preparation (Takuo Fujita et all, Heated Oyster Shell—Seaweed Calcium (AAA Ca) on Osteoporesis, Calcif. Issue Int. 58:226-230 (1996)).

The document FR 2 743 075 discloses a process for preparing biologically-active substances from mother-of-pearl, this process being essentially characterized by the fact that a hydrosoluble fraction is extracted from mother-of-pearl powder.

Finally, the homeopathic specialties CALCEREA OSTREICA and CALCAREA CARONICA OSTREARUM are known, both of which are oyster calcarea-based.

In this manner, the oyster shell and certain mother-of-pearl extracts are already proposed for therapeutic use, although this prior art concerns allopathic-dosed pharmaceutical compositions.

### SUMMARY OF THE INVENTION

Using the mother-of-pearl of marine mollusks and particularly the mother-of-pearl bio-aragonite from mollusks such as the Pinctada Maxima oyster in which the calcium carbonate is present in orthorhombic form, was never considered.

The corporate applicant found that this mother-of-pearl bio-aragonite offers significant therapeutic advantages, particularly in treating pain and more specifically in treating pain caused by pathologies and bone traumas.

The present invention relates therefore to a homeopathic medication characterized in that it includes at least one mother-of-pearl component, biocompatible mother-of-pearl bio-aragonite, at a minimal dilution of 1 DH. This medication is preferably used for treating bone pain and in particular pain associated with bone diseases caused by a pathology, pre-menopause or post-menopause estrogenic deficiency, ageing, cancer or trauma and to cartilaginous disorders particularly osteoarthritis.

### DETAILED DESCRIPTION OF THE INVENTION

Mother-of-pearl bio-aragonite is thus used, that is the mother-of-pearl including both its mineral and organic part, preferably in a micronized form. The bio-aragonite is made biocompatible through inhibition of the most immunogenic part of the bio-aragonite's organic substance in order to maintain only the organic substance bioactive in association with the mineral liable to have a positive effect.

Tests have been conducted on patients with osteoarthrosic pain (post-traumatic, diffuse, spinal osteoarthritis). Over a period of 60 days, these patients were nasally administered a biocompatible mother-of-pearl bio-aragonite-based homeopathic medication, diluted to 3DH, that is three successive one tenth dilutions, at the rate of one dose two times per week.

The development of the pain was then measured between days D0, D30 and D60 on a graduated pain scale ranging from 0 (no pain) to 10 (maximum pain).

These results are presented in table 1 below, which presents the development of the pain by 13 patients on days D0, D30 and D60 and the division factor of this pain between D0 and D60.

TABLE 1

Pain

| | | | | Pain division factor |
|---|---|---|---|---|
| Patient | D0 | D30 | D60 | D0-D60 |
| 1 | 9 | 6 | 3 | 3 |
| 2 | 10 | 8 | 4 | 2.5 |
| 3 | 7 | 5 | 5 | 1.4 |
| 4 | 7 | 5 | 2 | 3.5 |
| 5 | 8 | 6 | 4 | 2 |
| 6 | 7 | 5 | 3 | 2.33 |
| 7 | 7 | 5 | 3 | 2.33 |
| 8 | 6 | 4 | 2 | 2 |
| 9 | 7 | 3 | 6 | 1.16 |
| 10 | 8 | 6 | 7 | 1.14 |
| 11 | 9 | 5 | 1 | 9 |
| 12 | 9 | 6 | 2 | 4.5 |
| 13 | 7 | 4 | 3 | 2.33 |

These results thus show an average pain division factor between D0 and D60 of 2.86.

Only 3 of the 13 individuals have a pain division factor between DO and D60 less than 2.

These results thus highlight the efficiency of such homeopathic compositions.

Another object of the present invention is a process for preparing a homeopathic medication characterized by the fact that the selected mother-of-pearl components are diluted and/or suspended with a pharmaceutically acceptable excipient, which can consist in particular of an excipient or a solvent, and the mixture thus obtained is formulated.

This dilution is formulated in accordance with traditional techniques and quality standards implemented within the homeopathic industry. The homeopathic medications are then presented in a galenical form and packaged according to the desired method of administration, which may be oral, perlingual, nasal or in an injectable form.

More generally speaking, the medications have a dilution level from 1DH to 100K (KORSAKOFF).

Preferably, this level of dilution is between 1DH and 30CH (one-hundredth-dilution of mother tincture, thirty times in sequence), and even more preferably between 1DH and 1CH.

The examples below illustrate the invention, without limiting it.

### EXAMPLE 1

The excipient used is sucrose and the biocompatible bio-aragonite has a particle size of 50 to 150 microns.

One gram of bio-aragonite is diluted per 100 grams of sucrose, humid granulation is performed, then a sifting operation is performed to obtain the granules for perlingual administration.

### EXAMPLE 2

Under a laminar flow hood, 1 gram of biocompatible bio-aragonite is measured and mixed with 1 liter of sterile saline contained in a recipient which is opened at the last moment.

The recipient is then closed and the solution is stirred 20 times.

While still under the laminar flow hood, of this mother solution is sampled using a sterile syringe, which is then reinjected through a rubber stopper into a sterile bottle containing one liter of saline.

In this same bottle, using the same syringe which remained in place in the rubber stopper and another syringe, 1 ml of benzododecinium for 1 liter of preparation is added.

A solution in 2DH is thus obtained. This solution is then introduced into a spray bottle suitable for pernasal administration.

1. A homeopathic medication characterized in that it includes mother-of-pearl bio-aragonite which is rendered biocompatible through the inhibition of the most immunogenic part of the organic substance, at a minimal dilution of 1DH.

2. The homeopathic medication according to claim 1, presented in a galenical form for oral administration.

3. The homeopathic medication according to any one of claims 1 or 2, presented in a galenical form for perlingual administration.

4. The homeopathic medication according to claim 1, presented in a galenical form for parenteral administration.

5. The homeopathic medication according to claim 1, presented in a galenical form for nasal administration, particularly in the form of a spray.

6. A process for preparing the homeopathic medication according to any one of claims 1 to 5, characterized in that it consists in diluting and/or placing in suspension the biocompatible mother-of-pearl bio-aragonite using a pharmaceutical acceptable excipient which can namely consist of a diluting agent or a solvent, and to formulate the diluted mixture thus obtained.

7. The process for preparing the homeopathic medication according to claim 6, characterized in that the biocompatible bio-aragonite is used in pulverulent form, having a particle size between 50 and 150 microns.

8. The use of the biocompatible bio-aragonite for the preparation of a homeopathic medication for treating pain.

9. The use of the biocompatible mother-of-pearl bio-aragonite for the preparation of a homeopathic medication used for treating pain of bone origin and in particular related to bone diseases caused by a pathology, pre-menopause or post-menopause estrogenic deficiency, ageing, cancer or trauma and to cartilaginous disorders particularly osteoarthritis.
